(19) United States
(12) Patent Application Publication
(10) Pub. No.: US 2026/0334886 A1
(43) Pub. Date: Oct. 29, 2026

(54) METHOD AND SYSTEM FOR SCREENING CARDIAC ARRHYTHMIA

(71) Applicant: Turtle Shell Technologies Private Limited, Bengaluru (IN)

(72) Inventors: Gaurav PARCHANI, Bengaluru (IN); Ashwathi NAMBIAR, Bengaluru (IN); Sahil SINGH, Gandhinagar (IN); Jasmin Rishikesh CHAUGHULE, Mumbai (IN); Mudit DANDWATE, Bengaluru (IN)

(21) Appl. No.: 19/370,981

(22) Filed: Oct. 28, 2025

(30) Foreign Application Priority Data
Jul. 11, 2025 (IN) 202541066499

(51) Int. Cl. A61B 5/00; A61B 5/024; A61B 5/11

(52) U.S. Cl. CPC A61B 5/7275; A61B 5/02444; A61B 5/1102; A61B 5/6891; A61B 5/7264; A61B 5/7278

(57) ABSTRACT

The present disclosure provides a method for screening cardiac arrhythmia. The method comprises: acquiring vibrations-based physiological signals from a subject; processing the acquired vibrations-based physiological signals to extract a cardiac signal of the subject; estimating a heart rate and a heart rhythm from the extracted cardiac signal; identifying anomalies in at least one of: the estimated heart rate using a first set of metrics corresponding to heart rate, and the estimated heart rhythm using a second set of metrics corresponding to the heart rhythm; and detecting the cardiac arrhythmia based on the identified anomalies in at least one of: the estimated heart rate, the estimated heart rhythm.

### PRIORITY INFORMATION

The present application claims priority from Indian Patent Application Number 202541066499 filed on 11^{th} Jul. 2025.

### FIELD OF THE INVENTION

The present disclosure relates to a method for screening cardiac arrhythmia. The present disclosure also relates to a system for screening cardiac arrhythmia.

### BACKGROUND OF THE INVENTION

Cardiac health monitoring has garnered increasing clinical and technological interest due to the growing prevalence of cardiovascular disorders and the need for early detection and intervention. Amongst various cardiovascular disorders, cardiac arrhythmias, characterized by irregular heart rhythms, can range from benign to life-threatening. Early and accurate detection of cardiac arrhythmias is critical for effective management and prevention of complications such as stroke, heart failure, or sudden cardiac death. Traditionally, various cardiac health monitoring solutions such as electrocardiogram (ECG), Holter monitors, event recorders, echocardiography, electrophysiology studies (EPS), and blood tests are being employed for detection of cardiac arrhythmias.

However, the traditional cardiac health monitoring solutions come with notable limitations and are often constrained in their effectiveness and adaptability in detecting cardiac arrhythmias. The ECG provides only a brief snapshot and may miss intermittent cardiac arrhythmias, while Holter monitors offer limited recording durations and can be uncomfortable. Event monitors rely on patient recognition and activation during symptoms, making them ineffective for asymptomatic or brief episodes. Echocardiograms assess heart structure rather than electrical activity, and EPS, though precise, is invasive, costly, and not suitable for routine screening. Blood tests identify potential contributing factors but not cardiac arrhythmias directly. In other words, traditional cardiac health monitoring solutions struggle with detecting episodic and asymptomatic cardiac arrhythmias, often require clinical visits, depend on patient compliance, and generate large volumes of data that require time-consuming analysis. Moreover, the traditional cardiac health monitoring solutions predominantly rely on contact-based modalities such as leads or wearable monitoring devices which may cause discomfort to users/subjects during long-term monitoring.

Furthermore, the traditional cardiac health monitoring solutions are susceptible to signal artifacts caused by movement. Additionally, the traditional cardiac health monitoring solutions typically employ rule-based algorithms or fixed threshold criteria that fail to account for patient-specific variability and dynamic changes in cardiac behaviour over time. Furthermore, the traditional cardiac health monitoring solutions often lack the analytical granularity to detect subtle or transient irregularities, resulting in delayed or missed diagnoses. The limitations of existing solutions are further compounded in non-clinical environments, where consistent, reliable, and non-intrusive cardiac monitoring is essential for early risk stratification and long-term cardiac health management.

Therefore, in the light of foregoing discussion, there exists a need to overcome the aforementioned drawbacks.

### SUMMARY OF THE INVENTION

A primary objective of the present disclosure seeks to provide a method for screening cardiac arrhythmia that enables reliable and accurate detection of anomalies in heart rate and/or heart rhythm by analyzing vibrations-based physiological signals acquired using one or more non-contact sensors. The method facilitates extraction of cardiac signals and estimation of the heart rate and the heart rhythm, followed by identification of anomalies using defined metrics such as heart rate thresholds, heart rate variability, and entropy-based measures to detect potential arrhythmia. Another objective of the present disclosure seeks to provide a system for screening cardiac arrhythmia using the aforementioned method, comprising non-contact sensors and a processing unit configured to perform signal analysis and arrhythmia detection. An aim of the present disclosure is to provide a solution that overcomes at least partially the limitations of existing contact-based or complex diagnostic methods, thereby enabling non-intrusive, continuous, and effective cardiac monitoring.

In a first aspect, an embodiment of the present disclosure provides method for screening cardiac arrhythmia, the method comprising:
acquiring vibrations-based physiological signals from a subject;
processing the acquired vibrations-based physiological signals to extract a cardiac signal of the subject;
estimating a heart rate and a heart rhythm from the extracted cardiac signal;
identifying anomalies in at least one of:
the estimated heart rate using a first set of metrics corresponding to heart rate, and
the estimated heart rhythm using a second set of metrics corresponding to the heart rhythm; and
detecting the cardiac arrhythmia based on the identified anomalies in at least one of: the estimated heart rate, the estimated heart rhythm.

In a second aspect, an embodiment of the present disclosure provides a system for screening cardiac arrhythmia, the system comprising:
one or more non-contact sensors configured to acquire vibrations-based physiological signals from a subject; and
a processing unit operatively coupled to the one or more non-contact sensors, wherein the processing unit is configured to:
extract a cardiac signal of the subject from the acquired vibrations-based physiological signals received from the one or more non-contact sensors;
estimate a heart rate and a heart rhythm from the extracted cardiac signal;
identify anomalies in at least one of:
the estimated heart rate using a first set of metrics corresponding to heart rate, and
the estimated heart rhythm using a second set of metrics corresponding to the heart rhythm; and
detect the cardiac arrhythmia based on the identified anomalies in at least one of: the estimated heart rate, the estimated heart rhythm.

The aforementioned method and system are implemented for screening cardiac arrhythmia using vibrations-based physiological signals acquired through one or more non-contact sensors, minimizing any need for contact or injection to skin of a subject (e.g., a user, a person or a patient), and thereby eliminating the requirement for wearable devices or invasive diagnostic procedures. This enhances the subject's comfort and compliance, particularly during continuous or remote cardiac monitoring. Also, by implementing ways to monitor the subject via such one or more non-contact sensors, the aforementioned method and system allow continuous and non-invasive monitoring of cardiac function in even at home and ambulatory settings without repeated requirements to go to a clinical setting/medical facility. Moreover, the aforementioned method and system utilize mechanical vibration data captured by the non-contact sensors to extract cardiac signal waveforms, enabling accurate estimation of heart rate and heart rhythm without interfering with the subject's natural state. The aforementioned method and system further allow detailed analysis of heart rate metrics, rhythm irregularities, J-J intervals, and entropy-based characteristics to improve the quality and accuracy of arrhythmia detection. In other words, the system and method allow accurate detection of cardiac arrhythmia by analyzing unique inventive, and individualized custom features (such as changes in heart rate, heart rate variability, heart rhythm metrics, J-J intervals, and entropy-based measures extracted from physiological signals) allow for accurate and robust arrhythmia detection from non-contact vibration-based physiological signals. Advantageously, the system is configured to apply defined physiological metrics and optionally incorporate machine learning models for anomaly detection, confidence scoring, and classification of arrhythmia types, including atrial fibrillation, bradycardia, and tachycardia. This integration allows for enhanced diagnosis of potential cardiac arrhythmias while preserving a seamless, non-intrusive monitoring experience.

Embodiments of the present disclosure substantially eliminate or at least partially address the aforementioned problems in the prior art and facilitate accurate screening of cardiac arrhythmia using non-contact techniques.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of embodiments, is better understood when read in conjunction with the appended drawings. For the purpose of illustrating the present disclosure, exemplary constructions of the disclosure are shown in the drawings. However, the present disclosure is not limited to specific instrumentalities disclosed herein. Moreover, those skilled in the art will understand that the drawings are not to scale.

Embodiments of the present disclosure will now be described, by way of example only, with reference to the following diagrams wherein:

FIG. 1 illustrates a flowchart depicting steps of a method for screening cardiac arrhythmia, in accordance with an embodiment of the present disclosure;

FIG. 2A-2C illustrates schematic implementations depicting values a second set of metrics corresponding to heart rhythm calculated for screening cardiac arrhythmia, in accordance with an embodiment of the present disclosure;

FIG. 3 illustrates a scatter plot comparing Atrial Fibrillation (AF) and Normal Sinus Rhythm (NSR), in accordance with an embodiment of the present disclosure; and

FIG. 4 illustrates schematic illustration of an implementation of a system for screening cardiac arrhythmias, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

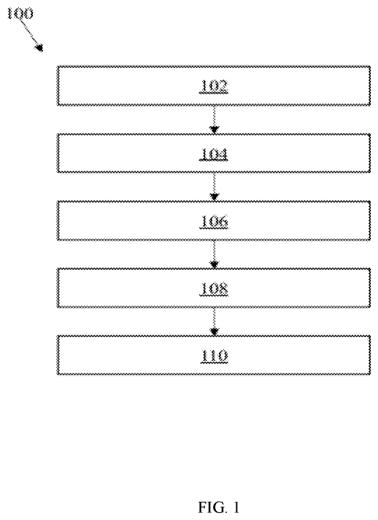

Referring to FIG. 1, illustrated is a flowchart 100 depicting steps of a method for screening cardiac arrhythmia, in accordance with an embodiment of the present disclosure. In this context, as shown in FIG. 1, at step 102, vibrations-based physiological signals are acquired from a subject. At step 104, the acquired vibrations-based physiological signals are processed to extract a cardiac signal of the subject. At step 106, a heart rate and a heart rhythm are estimated from the extracted cardiac signal. At step 108, anomalies in at least one of: the estimated heart rate using a first set of metrics corresponding to heart rate, and the estimated heart rhythm using a second set of metrics corresponding to the heart rhythm is identified. At step 110, the cardiac arrhythmia based on the identified anomalies in at least one of: the estimated heart rate, the estimated heart rhythm is detected.

In this regard, throughout the present disclosure, the term “cardiac arrhythmia” refers to any irregularity in the heart's rhythm, including but not limited to deviations from normal sinus rhythm, such as bradycardia (abnormally slow heart rate), tachycardia (abnormally fast heart rate), atrial fibrillation, premature atrial or ventricular contractions, and other rhythm disturbances. Notably, cardiac arrhythmia may manifest as irregular timing of heartbeats, inconsistent intervals between cardiac cycles, or abnormal waveform morphology, which may compromise effective cardiac output and pose significant health risks if undetected.

Throughout the present disclosure, the term “heart rate” refers to a physiological parameter representing a number of cardiac cycles (heartbeats) occurring within a defined unit of time. Typically, the heart rate is expressed in beats per minute (bpm). In other works, the heart rate reflects how often the subject's heart contracts to pump blood throughout the body and is a key indicator of cardiovascular function. Notably, the heart rate may vary based on factors such as age, fitness level, activity, medication use, and overall health, of the subject. Although, the heart rate can increase during physical activity, stress, or illness and decrease during rest or sleep, persistent abnormal heart rates such as the heart rate of a value which is too slow or too fast may indicate underlying cardiovascular disorders/conditions. Throughout the present disclosure, the term “heart rhythm” refers to patterns and regularity of the heartbeats and/or the cardiac cycles. It may be appreciated that the heart rhythm provides information on electrical impulses that coordinate the heart's contractions. Notably, a normal heart rhythm is known as a sinus rhythm, originates from the sinoatrial (SA) node, the heart's natural pacemaker, and maintains a steady, coordinated sequence of beats. An abnormal heart rhythm is observed when the heart rate is too fast or too slow. In such cases it may be concluded that the electrical impulses are irregularly generated driving the heart to contract abnormally suggesting possible cardiac disorders such cardiac arrythmia. Monitoring the heart rhythm is also crucial for detecting other cardiac disorders that may lead to symptoms like palpitations, dizziness, or even sudden cardiac arrest.

Throughout the present disclosure, the term “subject” refers to a person, patient, or animal whose cardiac activity is being monitored or assessed for potential arrhythmia. Notably, the subject may reside in a variety of settings, including but not limited to their own home, an assisted living facility, a nursing care center, a medical facility or a clinical research environment.

Throughout the present disclosure, the term “vibrations-based physiological signals” refers to data representing minute mechanical vibrations, oscillations, or displacements of the subject's body that arise due to underlying physiological processes, primarily associated with cardiac/cardiovascular movements/functions. The vibrations-based physiological signals are generated by internal biomechanical activities such as contraction and relaxation of heart muscle, ejection of blood into vascular system, valve closures, and recoil forces that occur during each cardiac cycle. The mechanical vibrations propagate through the body's tissues and skeletal structure as low-amplitude mechanical waves. For example, the vibrations-based physiological signals may be ballistocardiogram (BCG) signals. Referring to step 102, the vibrations-based physiological signals are acquired/captured from the subject using non-contact sensing mechanisms, such as one or more non-contact sensors. Optionally, the one or more non-contact based sensor may be at least one of: radar sensors, piezoelectric sensors, BCG sensors, or other suitable modalities capable of detecting motion without requiring direct skin contact.

Throughout the present disclosure, the term “cardiac signal” refers to a vibrational or mechanical waveform component of the vibrations-based physiological signals that corresponds to the mechanical activity of the heart during its pumping cycle. Notably, the cardiac signal comprises signals pertaining to subtle mechanical forces or vibrations generated by movements in the heart such as ventricular contraction, atrial filling, valve closures, and the propulsion of blood through arteries.

It may be appreciated that the vibrations-based physiological signals form the basis for extracting cardiac signal (performed at step 104) and by processing the vibrations-based physiological signals (acquired at step 102) using suitable filter and suitable filtering techniques. The suitable filter and the suitable filtering techniques are designed to isolate the cardiac signal by removing respiratory movements, gross body motions, ambient environmental noise and the likes. For instance, a bandpass filter (e.g., 0.5-20 Hz range) are applied to attenuate low-frequency components associated with respiration and motion artifacts, as well as high-frequency environmental noise. In addition to bandpass filter, the suitable filtering techniques may also include adaptive filtering, wavelet transform-based denoising, and empirical mode decomposition (EMD). Furthermore, blind source separation (BSS) methods such as, Independent Component Analysis (ICA), Principal Component Analysis (PCA), and Non-negative Matrix Factorization (NMF) are optionally applied to process the vibrations-based physiological signals. This allows for effective separation of the cardiac signal from respiratory components and motion-related artifacts. Optionally, the cardiac signal from the vibrations-based physiological signals is done through digital filters, autoencoders, or using a transformer model (such as a suitable neural network model) to perform required denoising, segmentation, and classification tasks to extract or reconstruct meaningful cardiac signals.

Referring to step 106, the extracted cardiac signal is analysed to extract information pertaining to the heart rate, and the heart rhythm. The estimation of the heart rate and the heart rhythm are achieved through a combination of signal processing techniques and machine learning (ML) models. For example, the signal processing techniques such as bandpass filtering, digital filtering, adaptive thresholding, peak detection, interval analysis, and template matching may be employed to estimate the heart rate and the heart rhythm of the subject from the cardiac signal. Furthermore, the machine learning models may be utilized to enhance the robustness and accuracy of heart rate and rhythm estimation by learning from labelled physiological signal datasets. The ML models can detect subtle waveform patterns and anomalies that may not be apparent through conventional signal processing techniques, thereby improving the reliability of cardiac assessment and enabling the detection of arrhythmic events.

Notably, the heart rate is derived from the periodicity of the cardiac signal. In this regard, the cardiac signal is processed to detect repetitive events such as ventricular contractions or other distinct mechanical events that correspond to individual heartbeats. Notably, the estimation of the heart rate may be performed using signal processing techniques such as peak detection, autocorrelation, or time-domain interval analysis. Furthermore, statistical techniques and machine learning (ML) models are employed to improve accuracy, filter out false positives caused by noise or artifacts, and adaptively handle variations in signal quality.

It may be appreciated that the heart rate and heart rhythm serve as a fundamental indicator of the subject's cardiorespiratory health, reflecting the autonomic regulation of the cardiovascular system. Variations in the heart rate, whether abnormally high (tachycardia), low (bradycardia), or irregular, may be indicative of cardiac arrhythmia or other cardiovascular dysfunctions. Similarly, variation in heart rhythm also indicates cardiac arrhythmia or other cardiovascular dysfunctions.

With reference to step 108, an identification of abnormalities in the heart rate may be performed based on the first set of metrics and the identification of abnormalities in the heart rhythm is performed by calculating the second set of metrics. Herein, the term “anomalies” refers to deviations or irregularities in the heart rate and/or the heart rhythm estimated from the cardiac signal that differ from expected normal patterns of cardiac activity. For example, the anomalies may include, but are not limited to, abnormal heart rates (e.g., bradycardia or tachycardia), irregular heart rhythms (e.g., cardiac arrhythmias), and atypical waveform features indicative of potential cardiovascular dysfunction. In simpler term, any deviating pattern in the heart rate and the heart rhythm of the subject may be considered as indicative of probable cardiac arrhythmia or other cardiovascular dysfunctions.

In this regard, throughout the present disclosure, the term “first set of metrics” refers to a group of quantitative parameters adapted to assess the heart rate information derived from the cardiac signal. It may be appreciated that the first set of metrics are indicative of the temporal and statistical characteristics of cardiac activity. The first set of metrics serve as primary indicators for assessing the stability, regularity, and variability of cardiac function, and are further used for detecting deviations or abnormalities in the subject's heart performance. It may be appreciated that the first set of metrics is used in combination with machine learning or statistical models for anomaly detection and the screening of cardiac arrhythmia.

In an embodiment, the first set of metrics corresponding to heart rate comprises at least one of:
a threshold range of heart rate; and
at least one statistical metric.

In this regard, the term “threshold range” used herein refers to a predefined ranged values of heart rate (i.e., a minimum and maximum acceptable heart rate values) within which the heart rate is expected to reside under normal or healthy conditions. For example, the threshold range may be defined as 60-100 bpm for the subject under monitoring. Herein, the heart rate below 60 bpm may indicate bradycardia and the heart rate exceeding 100 bpm may indicate tachycardia. By comparing the estimated heart rate (performed in step 106) against the threshold range, any possible deviations indicative of abnormal physiological states or anomalies in the heart rate can be identified. Such comparisons along with the at least one statistical metric forms a primary basis for anomaly detection in the heart rate. In this regard, the term “at least one statistical metric” refers to one or more quantitative measure used to summarize, describe, or evaluate the information/data pertaining to the estimated heart rate to assess variability or deviating patterns therein. The at least one statistical metric may comprise, but are not limited to, standard deviation, variance, coefficient of variation, mean absolute deviation, root mean square error (RMSE), correlation coefficient, outlier detection scores and any such suitable statistical metrics. It may be appreciated that the aforementioned statistical metrics can be used alone or in combination to accurately identify the anomalies in the estimated heart rate. The at least one statical metric is used to detect variations in heart rate that may not breach threshold boundaries but still reflect irregular behaviour. For example, a high standard deviation in the heart rate values over a short period may indicate atrial fibrillation or other arrhythmic conditions, even if individual heart rate readings remain within nominal limits. The technical advantages is accurate and reliable identification of anomalies in the heart rate.

Notably, the heart rhythm is estimated from the extracted cardiac signal, by evaluating consistency of inter-beat intervals and waveform morphology. Referring to step 108, any abnormalities in the heart rhythm (such as atrial fibrillation, premature ventricular contractions, or other arrhythmic patterns) are identified using at least one of: suitable signal processing techniques, machine learning techniques. The aforementioned suitable signal processing techniques and/or machine learning techniques are used to identify unique signal features from the cardiac signal morphology and anomalies in the heart rhythm therefrom. The suitable signal processing techniques may comprise at least one of: entropy measurement techniques, time-domain variability estimation techniques, digital filtering, template matching, adaptive thresholding and any such suitable signal processing techniques. The ML techniques may comprise rhythm classification models trained to distinguish between normal sinus rhythm and various types of cardiac arrhythmias. Notably, ML models are used to improve detection fidelity by learning complex patterns in vibrations-based physiological signals that may not be apparent through conventional rule-based approaches.

Throughout the present disclosure, the term “second set of metrics” refers to one or more analytical parameters derived from the cardiac signal, which are used to characterize and quantify nature and extent of the anomalies in the heart rhythm. The second set of metrics facilitates enhanced anomaly detection in heart rhythm (which is more complex than identifying anomalies in the heart rate) by evaluating deviating patterns, distribution, and confidence of detected irregularities in cardiac activity. The second set of metrics may include, but are not limited to, abnormal rhythm density, anomaly duration, anomaly frequency, statistical confidence scores, entropy values, temporal irregularity indices, and machine learning-based anomaly scores. It may be appreciated that the second set of metrics enables more refined classification and differentiation between types of cardiac arrhythmias, providing a deeper level of diagnostic insight. The second set of metrics may be used by rule-based engines or machine learning models to assign severity levels or trigger alerts for potential cardiac risk.

In an embodiment, the second set of metrics corresponding to the heart rhythm comprises at least one of:
heart rate variability metric is calculated based on intervals between successive J peaks in the cardiac signal, and wherein a threshold for interval difference between the successive J peaks is dynamically determined based on the subject's heart rate;

Shannon entropy of J-J intervals calculated from time intervals between the successive J peaks in the cardiac signal;
Shannon Entropy of J-J interval differences calculated based on a difference between successive J-J time interval;
interval between specific waveform peaks in the cardiac signal;
a presence or an absence of specific waveform peaks in the cardiac signal; and
morphology and waveform characteristics of the cardiac signal.

In this regard, the term “heart rate variability metric” refers to a parameter that quantifies the physiological variation in the time interval between successive heartbeats, as derived from the cardiac signal. Specifically, the heart rate variability (HRV) metric is calculated based on the intervals between successive J peaks identified within the cardiac signal. Herein, the term “J peaks” refers to distinct mechanical events corresponding to ventricular ejection in each cardiac cycle. For example, the J peaks depict a waveform at a time of complete contraction of the heart. The HRV metric captures beat-to-beat variability and is used to assess cardiac health, and susceptibility to cardiac arrhythmias. It may be appreciated that the threshold for the interval difference between successive J peaks is dynamically determined based on the subject's heart rate, allowing for adaptive and personalized anomaly detection. For instance, as the average heart rate increases, acceptable variability between J-J intervals may reduce, and vice-versa. The dynamic determination of the threshold enhances reliability of detecting abnormal cardiac rhythm patterns.

Notably, to estimate anomalies in heart rhythm, a metric called the percentage of successive J-J intervals that differ by more than x milliseconds (pJJx) is computed, where ‘x’ is a unit of time, typically expressed using milliseconds. This metric is calculated by dividing the number of successive J-J interval differences greater than ‘x’ milliseconds by the total number of J-J intervals. The J-J intervals represent the time between successive normal J-peaks in vibration-based physiological signals (i.e., the BCG signals) acquired from the subject. This parameter is commonly used as a clinical measure to quantify heart rate variability (HRV). The value of x is individualized i.e., adjusted based on the subject's estimated heart rate. For example, a person with a heart rate of around 60 bpm typically has J-J intervals of approximately XX ms, while someone with a heart rate of 100 bpm has J-J intervals around YY ms. Therefore, a change of ZZ ms or more in successive J-J intervals is more significant for someone with a faster heart rate. Selecting an appropriate value of ‘x’, typically one that is inversely proportional to heart rate, enables more accurate and meaningful comparison of pJJx across different subjects or time points.

In this regard, the term “Shannon entropy” refers to a statistical measure of randomness or unpredictability in a degree of irregularity/variability in the time intervals between cardiac events such as, the J-J intervals between two consecutive J peaks and/or J-J interval differences calculated between successive J-J intervals. In other words, Shannon entropy of J-J intervals and Shannon Entropy of J-J interval differences are calculated in order to quantify disorder, uncertainty, or randomness in the heart rhythm. Higher value of the Shannon entropy indicates greater variability and potential irregularity in the heart rhythm, while lower entropy values reflect more regular and predictable heart patterns.

Notably, the Shannon entropy is computed by evaluating a probability distribution of observed values (e.g., J-J intervals or their differences) and summing the negative product of each probability with its logarithm. For instance, the Shannon entropy of J-J intervals is calculated based on the time intervals between successive J peaks in the cardiac signal. Herein, the Shannon entropy of J-J intervals metric captures how uniformly or variably durations between heartbeats are distributed. A relatively constant J-J interval across cycles would yield low entropy, indicating a regular heart rhythm, while more irregular distributions may yield higher entropy.

Additionally, the Shannon entropy of J-J interval differences is calculated based on the variation between successive J-J time intervals, that is, the difference in duration in duration from one J-J interval to next duration from successive J-J intervals. The Shannon entropy of J-J interval differences reflects second-order variability, capturing abrupt changes in rhythm that might not be evident in the J-J interval alone. Measuring entropy in the Shannon entropy of J-J interval differences allows detection of fine-grained fluctuations in the heart rhythm.

For an example, a sequence of ten successive heartbeat time instants (in milliseconds) is detected from the cardiac signal segment beginning at time t. Let the detected time instants corresponding to J-peaks be represented as:

a₁=[1000, 1750, 2480, 3300, 3950, 4700, 5550, 6200, 7050, 7680]

Based on these values, the J-J intervals, representing the time intervals between successive J-peaks, are computed as:

a₂=[1750-1000, 2480-750, 3300-2480, 3950-3300, 4700-3950, 5550-4700, 6200-5550, 7050-6200, 7680-7050]=[750, 730, 820, 650, 750, 850, 650, 850, 630]

Now, the Shannon entropy of J-J intervals is calculated over the vector α₂.

Subsequently, the differences between successive J-J intervals are derived to assess beat-to-beat variability in the heart rhythm. The resulting vector is:

a₃=[730-750, 820-730, 650-820, 750-650, 850-750, 650-850, 850-650, 630-850]=[−20, 90, −170, 100, 100, −200, 200, −220]

herein, the Shannon entropy of J-J interval differences is computed over this derived vector α₃. This metric reflects the irregularity in the differences between consecutive J-J intervals and provides insights into transient arrhythmic patterns that may not be evident from absolute J-J intervals alone.

Further, the second set of metrics also include the interval between specific waveform peaks in the cardiac signal. Herein the term “specific waveforms peaks” refers to identified characteristic peaks in the cardiac signal waveform, including peaks labelled F, G, H, I, J, K, L, M, N, as observed in the vibration-based physiological signals i.e., the BCG signal. The specific waveform peaks correspond to specific mechanical events of the cardiac cycle such as isovolumetric contraction, ejection, valve closure, and ventricular filling. Notably, the specific waveform peaks are detected using algorithms-based signal processing and/or the machine learning models trained to recognize such specific waveform peaks. It may be appreciated that, the time intervals between these peaks (e.g., I-J, J-K, or broader intervals such as J-J (successive cardiac cycles)) provide quantitative markers for evaluating the mechanical timing of the heart rhythm. The presence or the absence of the specific waveform peaks in the cardiac signal are indicative of underlying hemodynamic events, and their consistent presence across cardiac cycles is typically associated with normal heart function. Absence, attenuation, or distortion of the specific waveform peaks may indicate cardiac anomalies such as arrhythmia. The technical advantage is accurate and reliable detection of anomalies in the heart rhythm can indicate minute level cardiac arrhythmia.

Moreover, to determine anomalies in heart rhythm, the morphology and waveform characteristics of the cardiac signal are analysed. The term “morphology” refers to shape of the cardiac signal including notching, flattening, sharpness and so on which when analysed provide information on cardiac health and aids in distinguishing arrhythmias in the subject. The term “waveform characteristic” refers to various features and patterns present in the cardiac signal such as amplitude, wavelength, frequency, segments, intervals and so on. The waveform characteristics are crucial for identifying normal and abnormal heart function and aid in diagnosis of cardiac arrhythmias. The morphology and waveform characteristics are analysed to characterize cardiac function and provide insight into any anomalies in heart rate and heart rhythm. In this regard, amplitude metrics such as peak-to-peak heights of the I-J-K complex, height of other peaks reflect cardiac stroke volume and contractile force involved in heart/cardiac functionality. For example, the amplitudes of I and/or J peaks are directly proportional to stroke volume and diminished amplitude may indicate I and/or J peaks reduced cardiac output or weakened contraction. Time intervals between peaks measure cardiac timing. For example, the duration of the I-K complex or H-K span estimates left ventricular ejection time, while the H-I interval and M-N interval correspond to isovolumic contraction and relaxation phases, respectively. When the cardiac functionalities are compromised or any possible cardiac conditions are present, then these intervals are abnormally prolonged or shortened. Similarly, from frequency data (extracted from of the cardiac signal) anomalies in heart rate and heart rhythm can be identified. So, from analysing (such as using short-time Fourier or wavelet transforms) the morphology, amplitude, time interval, frequency and so on arrhythmic irregularity can be detected.

Referring to step 110, based on the anomalies detected in the heart rate and/or the heart rhythm corresponding to the subject under observation/monitoring, a possibility of the cardiac arrhythmia is calculated.

In an embodiment, the method further comprises using a machine learning model to identify anomalies in at least one of:
a. the estimated heart rate using the first set of metrics corresponding to heart rate, and
b. the estimated heart rhythm using the second set of metrics corresponding to the heart rhythm; and
detect the cardiac arrhythmia based on the identified anomalies in at least one of: the estimated heart rate, the estimated heart rhythm.

In this regard, throughout the present disclosure, the term “machine learning (ML) model” refers to a model that is trained to recognize patterns, extract features, calculate various metrics (such as the first set of metrics and the second set of metrics), compare extracted data on heart rate and heart rhythm, identify anomalies to detect cardiac arrhythmias without being explicitly programmed with rule-based logic. The ML model is configured to operate on vibrations-based physiological signals, particularly the cardiac signal, to perform one or more tasks such as detecting anomalies in heart rate and/or the heart rhythm, identifying waveform peaks (e.g., J peaks), classifying types of arrhythmia, or assigning confidence scores to the presence of such anomalies. The ML model may be trained on labelled or unlabelled datasets using supervised, unsupervised, or semi-supervised learning techniques. Examples of suitable machine learning models include, but are not limited to, support vector machines (SVMs), random forests, k-nearest neighbours (k-NN), decision trees, logistic regression, and neural networks (e.g., convolutional neural networks (CNNs) or recurrent neural networks (RNNs)). In certain embodiments, ensemble models or deep learning architectures may be utilized to enhance detection accuracy and robustness.

Notably, the machine learning model is used for identifying the specific waveform peaks such as F, G, H, I, J, K, L, M, N peaks from the BCG signals, estimating the heart rate and the heart rhythm, distinguishing normal patterns from arrhythmic or irregular cardiac patterns, and generating a confidence score representing the likelihood of the detected anomaly or classification. The technical advantage is that it enables adaptive, data-driven decision-making, facilitating continuous learning and improving performance over time as well accurate screening of the cardiac arrhythmia.

In an embodiment, the machine learning model is further configured to generate a confidence score for the cardiac arrhythmia, based on a repeatability data of detected anomalies in at least one of: the estimated heart rate, the estimated heart rhythm, across multiple cardiac cycles. In this regard, the term “confidence score” refers to a numerical or categorical measure generated by the machine learning model that represents statistical likelihood, reliability, or certainty of the detected cardiac arrhythmia being a true positive finding, rather than a false positive resulting from noise, motion artifact, or transient variation. The confidence score is derived from evaluating consistency and persistence of the detected anomalies such as irregular heart rhythm or abnormal heart rate. It may be appreciated that, the confidence score is presented as a scalar value (e.g., between 0 and 1) or a discrete class (e.g., “low confidence,” “moderate confidence,” or “high confidence”). Moreover, the confidence score may be calibrated (or set to a predefined limit) to trigger alerts or initiate further diagnostic processing. The technical advantage is that the confidence score enables improved clinical relevance and interpretability of the arrhythmia detection, reducing false alarms and supporting decision-making in remote or continuous monitoring environments.

In an embodiment, the method further comprises classifying the cardiac arrhythmia type selected from at least one of: atrial fibrillation, atrial flutter, premature atrial contractions, premature ventricular contractions, bradycardia, tachycardia. In this regard, the classification of the cardiac arrhythmia is performed based on analysis of the extracted cardiac signal and the detected anomalies in heart rate and the heart rhythm. The classification may utilize rule-based logic, statistical metrics, or trained machine learning models configured to differentiate among distinct arrhythmia patterns based on characteristic features present in the cardiac waveform.

Herein, the term “atrial fibrillation” refers to a type of arrhythmia characterized by rapid and irregular beating of the atrial chambers, often resulting in absence of identifiable P-waves or J-peaks, and highly irregular intervals between successive heartbeats. It may be inferred from irregular J-J intervals and elevated entropy metrics. In other words, the atrial fibrillation can be defined as an irregular and often rapid heartbeat that originates in the upper chambers (i.e., atria) of the heart accompanied by physical symptoms such as palpitations, dizziness or light-headedness, shortness of breath, fatigue, and weakness, in the subject. The atrial fibrillation may lead to blood clots, stroke, and heart failure, if not diagnosed properly.

The term “atrial flutter” refers to a type of cardiac arrhythmia that is marked by rapid but regular atrial contractions, leading to a “sawtooth” morphological pattern in the cardiac signal and recurring waveform peaks at regular but high-frequency intervals. It may present as repetitive, uniform oscillations within the cardiac signal with abnormal heart rhythm. The atrial flutter can be caused by various factors, including high blood pressure, heart valve disease, or structural heart changes and a proper diagnostics thereof is essential in early and efficient treatment of aforementioned cardiac dysfunctions.

The term “premature atrial contractions” refers to early heartbeats originating in atria of the heart, detected as out-of-sequence waveform peaks with shortened intervals followed by compensatory pauses. Notably, in this case, the morphology of the cardiac signal shows presence of additional peaks.

The term “premature ventricular contractions” refers to a type of cardiac arrhythmia that originates in ventricles of the heart and is detected as widened or abnormally shaped waveform components in the cardiac signal, typically interrupting the normal rhythm and followed by a compensatory pause.

The technical advantage is that the classification facilitates targeted alerts, clinical triage, and condition-specific responses in automated cardiac monitoring systems, thereby making the diagnosis efficient.

In an embodiment, the method further comprises storing data pertaining to the acquired vibrations-based physiological signals, the extracted cardiac signal, the estimated heart rate, the estimated heart rhythm, the first set of metrics, and the second set of metrics in a database. Herein, the term “database” refers to structured collection of data that is stored and managed using a computing system, and that allows for efficient querying, retrieval, updating, and organization of information. The database may include, but is not limited to, relational databases (e.g., SQL-based), non-relational databases (e.g., NoSQL, key-value stores, document stores), vector databases, time-series databases, or object-oriented databases. The database may reside locally on a computing device, be distributed across multiple nodes or systems, or be implemented in a cloud-based or edge computing environment. The data stored in the database can be accessed remotely by an authorized user such as a physician in charge of the subject, a healthcare provider, a researcher and so on. the data pertaining to the acquired vibrations-based physiological signals, the extracted cardiac signal, the estimated heart rate, the estimated heart rhythm, the first set of metrics, and the second set of metrics can be stored in the database using a suitable network means, a transferable drive or any such suitable transfer medium. The technical advantages of storing data in the database are achieving data security, providing a data backup and promoting remote monitoring.

In an embodiment, the method further comprises transmitting data pertaining to the detected cardiac arrhythmia to at least one of: a user interface, a remote server, for further review. In this regard, the term “user interface” refers to an input/output interface comprising hardware and software components configured to receive and transmit various instructions, observations and so on. It may be appreciated that the user interface is configured to present physiological monitoring data, alerts, and diagnostic outputs to the user in a comprehensible format such as in graphical plots, in tabulation forms (spreadsheets), text messages, siren/alarm, blinking lights and so on. Moreover, the user interface may be implemented as part of a smartphone application, a dedicated health monitoring dashboard, a wearable device display, or a web-based portal, and is designed to visually or audibly convey arrhythmia detection outcomes, trend data, and actionable notifications to the subject or authorized personnel. In this regard, the term “remote server” refers to a computing system or network-connected storage infrastructure located remotely from the subject or sensing device, configured to receive, store, process, or analyze physiological monitoring data. The remote server may be part of a centralized health management platform, a cloud-based analytics engine, or an electronic health record (EHR) system. The transmission facilitates timely access to diagnostic insights by relevant stakeholders including the subject, caregivers, or medical professionals. The technical advantage is that it enables asynchronous access to historical data, application of additional computational models, and integration with telehealth services for further clinical interpretation and decision support.

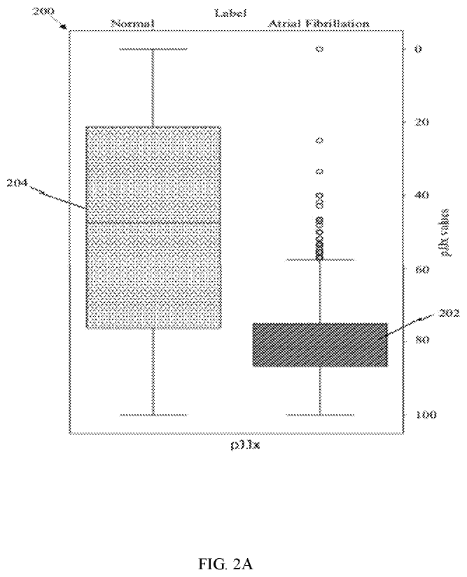

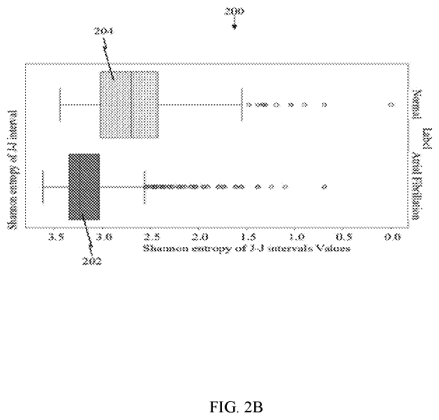

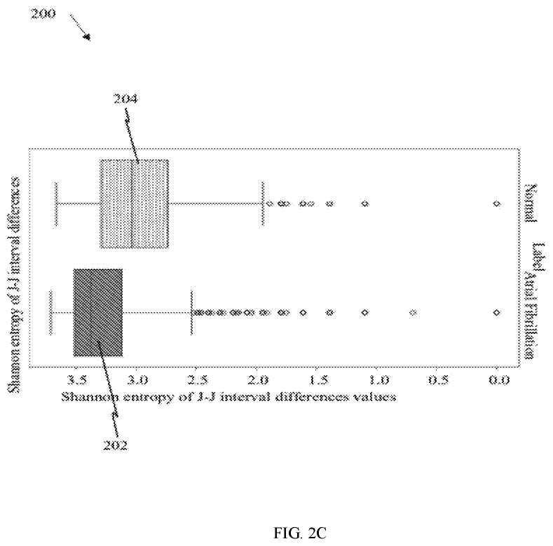

With reference to FIG. 2A-2C, illustrated are schematic illustrations 200 of the second set of metrics corresponding to the heart rhythm for screening cardiac arrhythmia, in accordance with an embodiment of the present disclosure. Each sub-figure (FIGS. 2A, 2B and 2C) presents a comparative box plot analysis between cardiac segments labelled as Atrial Fibrillation (AF) class 202 and Normal class 204. The AF class 202 is represented using a darker shade (grey hatch), and the Normal class 204 is represented using a lighter shade (white hatch).

With reference to FIG. 2A, the plot is a heart rate variability (HRV) measure, specifically denoted as pJJx. It is observed that the AF class 202 show a significantly higher median and interquartile range compared to Normal class 204, indicating greater beat-to-beat variability of the HRV commonly associated with atrial fibrillation. The Normal class 204 exhibit tighter distribution and lower variability, reflecting more regular heart rhythm. With reference to FIG. 2B, the Shannon entropy of J-J intervals is depicted. The AF class 202 herein exhibit higher entropy values relative to the Normal class, with elevated median and reduced lower outliers. This suggests that timing between successive J-J interval in AF class 202 is less predictable and more disordered than in regular heart rhythms while compared to the Normal class 204. With reference to FIG. 2C, the Shannon entropy of J-J intervals differences is illustrated. The plot herein depicts the AF class 202 showing higher entropy levels in contrast to the Normal class 204. This further reinforces presence of irregularity and increased heart rhythm complexity in arrhythmic conditions. Collectively, the FIG. 2A, FIG. 2B and FIG. 2C representing the heart rate variability (pJJx), the Shannon entropy of J-J intervals, and Shannon entropy of J-J interval differences respectively demonstrate statistically distinguishable patterns between arrhythmic heart rhythms and normal heart rhythm.

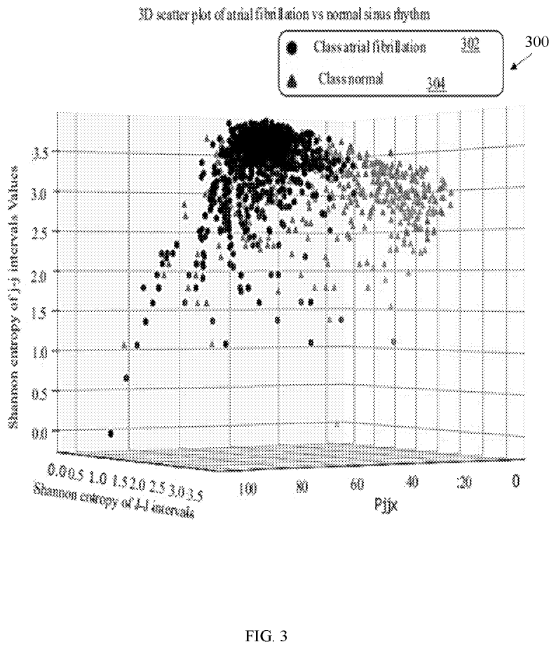

With reference to FIG. 3, illustrated is a scatter plot 300 comparing Atrial Fibrillation (AF) and Normal Sinus Rhythm (NSR), in accordance with an embodiment of the present disclosure. The plot comprises three axes namely: X-axis representing Shannon entropy of J-J interval, Y-axis representing pJJx (a measure of heart rate variability), and Z-axis representing Shannon entropy of J-J interval differences. Herein, data points corresponding to the AF are shown as black circles as class AF 302, and data points corresponding to the Normal class are shown as grey triangles as class normal 304. The FIG. 3 representing multidimensional feature space demonstrates strong separability between heart rhythms of the AF class 302 and the Normal class 304. For example, points relating to AF class 302 are concentrated in the upper-right region, characterized by higher entropy values along both axes (Shannon entropy of J-J interval and Shannon entropy of J-J interval differences) and elevated HRV (pJJx) values. This reflects increased irregularity and complexity in beat-to-beat timing and its variability, which is characteristic of arrhythmic cardiac patterns. Conversely, points relating to normal class 304 are clustered in the lower-left region, showing lower Shannon entropy and reduced HRV, indicative of a more regular and predictable heart rhythm. The distinct clustering observed in the multidimensional feature space supports effectiveness of combining entropy-based metrics and HRV measures for robust classification of the heart rhythm.

It may be appreciated that the information disclosed under FIGS. 2A-2C and FIG. 3 are to be read and understood in conjunction with steps described under disclosure of FIG. 1.

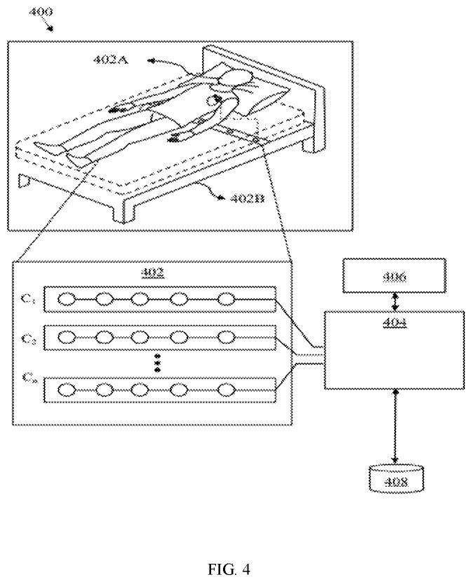

With reference to FIG. 4, illustrated is a schematic illustration of an implementation of a system 400 for screening cardiac arrhythmia, in accordance with an embodiment of the present disclosure. As shown, the system 400 comprises one or more non-contact sensors 402, a processing unit 404, a user interface 406 and a database 408. It may be appreciated that the system 400 is configured to use the aforementioned method for screening cardiac arrhythmia.

In this context, the processing unit 404 refers to an electronic component in of the system 400 that is configured to executes specific functions necessary for operation of the system 400. Notably, the processing unit 404 may be a microcontroller, micro-processing unit, an on-chip control unit, a central processing unit, or any such suitable arrangement capable of receiving input, analyse the input received, and perform required operation thereafter. In an embodiment, the processing unit 404 maybe onboard the user interface 406. In other words, the processing unit 404 may be s integrated directly into the user interface 406 rather than being a separate component.

In an embodiment, the system 400 is implemented in an environment designed for continuous, non-contact screening of cardiac arrhythmia, comprising, a support surface 402B wherein the one or more non-contact sensors 402 may be embedded. Notably, the support surface 402B serves as a physical structure on which the subject 402A rests, facilitating the detection of mechanical forces generated by breathing movements. While the support surface 402B is typically a bed, it may also be a sofa, orthopaedic mattress, chair, or any surface where the torso of the subject's 402A body can be rested, enabling flexible deployment across various settings such as home environments, hospitals, sleep clinics, and assisted living facilities.

Since the one or more non-contact sensors 402 is positioned beneath the mattress or bedding, the system 400 remains entirely non-contact, making it comfortable for continuous monitoring in both clinical and home settings. The support surface 402B (bed) serves as a stable medium for transmitting mechanical forces, allowing the one or more non-contact sensors to vibrations-based physiological signals from the subject 402A without requiring direct attachment to the subject 402A. The non-invasive nature of the sensor placement eliminates the need for wearable belts or intrusive monitoring equipment, reducing user discomfort and improving compliance for long-term respiratory health tracking. Notably, the subject 402A lies on the support surface 402B, with their torso positioned above the one or more non-contact sensors 402, which are placed beneath the mattress or bedding or cushioning placed on the support surface. This arrangement enables the system 400 to acquire the vibrations-based physiological signals generated by the subject's cardiac/cardiovascular movements/functions, in a non-contact manner.

Notably, the processing unit 404 is configured to executed steps of the aforementioned method for screening cardiac arrhythmia that the subject 402A may have. In this regard, the processing unit 404 is operatively connected to the one or more non-contact sensors 402. Moreover, the processing unit 404 is also configured to extract the cardiac signal of the subject 402A. The processing unit 404 is also configured to estimate heart rate and a heart rhythm from the extracted cardiac signal. Further, the processing unit 404 is configured to identify anomalies in at least one of: the estimated heart rate using a first set of metrics corresponding to heart rate, and the estimated heart rhythm using a second set of metrics corresponding to the heart rhythm. Furthermore, the processing unit 404 is configured to detect the cardiac arrhythmia based on the identified anomalies in at least one of: the estimated heart rate, the estimated heart rhythm.

It may be appreciated that the processing unit 404 is also communicably coupled to the database 408 for receiving additional input, for example, the processing unit 404 is configured to store data pertaining to the acquired vibrations-based physiological signals, the extracted cardiac signal, the estimated heart rate, the estimated heart rhythm, the first set of metrics, and the second set of metrics. The data pertaining to the detected cardiac arrhythmia can be transmitted to a user interface 406 for the medical professional to review. The user interface 406 is at least one of: a smartphone application, a tablet, a desktop dashboard, or an integrated hospital monitoring system, allowing both the subject 402A and the medical professionals to access real-time respiratory insights.

The system 400 further includes the database 408, which may be a cloud-based database, local encrypted database, or hospital server, ensuring secure, long-term data retention. This enables the system 400 to track historical heart rate and heart rhythm, monitor disease progression, and support AI-driven predictive analysis for early intervention in screening of cardiac arrhythmia.

In an embodiment, the first set of metrics corresponding to heart rate comprises at least one of:
a threshold range of heart rate;
and
at least one statistical metric.

In an embodiment, the second set of metrics corresponding to the heart rhythm comprises at least one of:
heart rate variability metric is calculated based on intervals between successive J peaks in the cardiac signal, and wherein a threshold for interval difference between the successive J peaks is dynamically determined based on the subject's heart rate;
Shannon entropy of J-J intervals calculated from time intervals of the successive J peaks in the cardiac signal;
Shannon entropy of J-J interval differences calculated based on a difference between successive J-J interval;
interval between specific waveform peaks in the cardiac signal;
a presence or an absence of specific waveform peaks in the cardiac signal; and
morphology and waveform characteristics of the cardiac signal.

In an embodiment, the processing unit is further configured to use a machine learning model to identify anomalies in at least one of:
c. the estimated heart rate using the first set of metrics corresponding to heart rate, and
d. the estimated heart rhythm using the second set of metrics corresponding to the heart rhythm; and
   detect the cardiac arrhythmia based on the identified anomalies in at least one of: the estimated heart rate, the estimated heart rhythm.

In an embodiment, the processing unit is further configured to use the machine learning model to generate a confidence score for the cardiac arrhythmia, based on a repeatability data of detected anomalies in at least one of: the estimated heart rate, the estimated heart rhythm, across multiple cardiac cycles.

In an embodiment, the processing unit is further configured to use the machine learning model to classify the cardiac arrhythmia type selected from at least one of: atrial fibrillation, atrial flutter, premature atrial contractions, premature ventricular contractions, bradycardia, tachycardia.

In an embodiment, the processing unit is further configured to store data pertaining to the acquired vibrations-based physiological signals, the extracted cardiac signal, the estimated heart rate, the estimated heart rhythm, the first set of metrics, and the second set of metrics, in a database.

In an embodiment, the processing unit is further configured to transmit data pertaining to the detected cardiac arrhythmia to at least one of: a user interface, a remote server, for further review.

It may be appreciated that various embodiments and variants disclosed above, with respect to the aforementioned method, apply mutatis mutandis to the system 400 as well.

Modifications to embodiments of the invention described in the foregoing are possible without departing from the scope of the invention as defined by the accompanying claims. Expressions such as “including”, “comprising”, “incorporating”, “consisting of”, “have”, “is” used to describe and claim the present invention are intended to be construed in a non-exclusive manner, namely allowing for items, components or elements not explicitly described also to be present. Reference to the singular is also to be construed to relate to the plural. Numerals included within parentheses in the accompanying claims are intended to assist understanding of the claims and should not be construed in any way to limit subject matter claimed by these claims.

1. A method for screening cardiac arrhythmia, the method comprising:
acquiring, via one or more non-contact sensors comprising at least one sensor selected from radar sensors, piezoelectric sensors, and ballistocardiogram (BCG) sensors, vibrations-based physiological signals from a subject;
processing the acquired vibrations-based physiological signals to extract a cardiac signal of the subject;
estimating a heart rate and a heart rhythm from the extracted cardiac signal;
identifying anomalies in at least one of:
the estimated heart rate using a first set of metrics corresponding to heart rate, and
the estimated heart rhythm using a second set of metrics corresponding to the heart rhythm; and
detecting the cardiac arrhythmia based on the identified anomalies in at least one of:
the estimated heart rate, the estimated heart rhythm.

2. The method as claimed in claim 1, wherein the first set of metrics corresponding to heart rate comprises at least one of:
a threshold range of heart rate; and
at least one statistical metric.

3. The method as claimed in claim 1, wherein the second set of metrics corresponding to the heart rhythm comprises at least one of:
heart rate variability metric is calculated based on intervals between successive J peaks in the cardiac signal, and wherein a threshold for interval difference between the successive J peaks is dynamically determined based on the subject's heart rate;
Shannon entropy of J-J intervals calculated from time intervals between the successive J peaks in the cardiac signal;
Shannon Entropy of J-J interval differences calculated based on a difference between successive J-J time interval;
interval between specific waveform peaks in the cardiac signal;
a presence or an absence of specific waveform peaks in the cardiac signal; and
morphology and waveform characteristics of the cardiac signal.

4. The method as claimed in claim 1, further comprises using a machine learning model to identify anomalies in at least one of:
the estimated heart rate using the first set of metrics corresponding to heart rate, and
the estimated heart rhythm using the second set of metrics corresponding to the heart rhythm; and
detect the cardiac arrhythmia based on the identified anomalies in at least one of: the estimated heart rate, the estimated heart rhythm.

5. The method as claimed in claim 4, wherein the machine learning model is further configured to generate a confidence score for the cardiac arrhythmia, based on a repeatability data of detected anomalies in at least one of: the estimated heart rate, the estimated heart rhythm, across multiple cardiac cycles.

6. The method as claimed in claim 1, further comprising classifying the cardiac arrhythmia type selected from at least one of: atrial fibrillation, atrial flutter, premature atrial contractions, premature ventricular contractions, bradycardia, tachycardia.

7. The method as claimed in claim 1, further comprising storing data pertaining to the acquired vibrations-based physiological signals, the extracted cardiac signal, the estimated heart rate, the estimated heart rhythm, the first set of metrics, and the second set of metrics in a database.

8. The method as claimed in claim 1, further comprising transmitting data pertaining to the detected cardiac arrhythmia to at least one of: a user interface, a remote server, for further review.

9. A system for screening cardiac arrhythmia, the system comprising:
one or more non-contact sensors configured to acquire vibrations-based physiological signals from a subject; and
a processing unit operatively coupled to the one or more non-contact sensors, wherein the processing unit is configured to:
extract a cardiac signal of the subject from the acquired vibrations-based physiological signals received from the one or more non-contact sensors;
estimate a heart rate and a heart rhythm from the extracted cardiac signal;
identify anomalies in at least one of:
the estimated heart rate using a first set of metrics corresponding to heart rate, and
the estimated heart rhythm using a second set of metrics corresponding to the heart rhythm; and
detect the cardiac arrhythmia based on the identified anomalies in at least one of: the estimated heart rate, the estimated heart rhythm.

10. The system as claimed in claim 9, wherein the first set of metrics corresponding to heart rate comprises at least one of:
a threshold range of heart rate; and
at least one statistical metric.

11. The system as claimed in claim 9, wherein the second set of metrics corresponding to the heart rhythm comprises at least one of:
heart rate variability metric is calculated based on intervals between successive J peaks in the cardiac signal, and wherein a threshold for interval difference between the successive J peaks is dynamically determined based on the subject's heart rate;
Shannon entropy of J-J intervals calculated from time intervals of the successive J peaks in the cardiac signal;
Shannon entropy of J-J interval differences calculated based on a difference between successive J-J interval;
interval between specific waveform peaks in the cardiac signal;
a presence or an absence of specific waveform peaks in the cardiac signal; and
morphology and waveform characteristics of the cardiac signal.

12. The system as claimed in claim 9, wherein the processing unit is further configured to use a machine learning model to identify anomalies in at least one of:
the estimated heart rate using the first set of metrics corresponding to heart rate, and
the estimated heart rhythm using the second set of metrics corresponding to the heart rhythm; and
detect the cardiac arrhythmia based on the identified anomalies in at least one of: the estimated heart rate, the estimated heart rhythm.

13. The system as claimed in claim 12, wherein the processing unit is further configured to use the machine learning model to generate a confidence score for the cardiac arrhythmia, based on a repeatability data of detected anomalies in at least one of: the estimated heart rate, the estimated heart rhythm, across multiple cardiac cycles.

14. The system as claimed in claim 12, wherein the processing unit is further configured to use the machine learning model to classify the cardiac arrhythmia type selected from at least one of: atrial fibrillation, atrial flutter, premature atrial contractions, premature ventricular contractions, bradycardia, tachycardia.

15. The system as claimed in claim 9, wherein the processing unit is further configured to store data pertaining to the acquired vibrations-based physiological signals, the extracted cardiac signal, the estimated heart rate, the estimated heart rhythm, the first set of metrics, and the second set of metrics, in a database.

16. The system as claimed in claim 9, wherein the processing unit is further configured to transmit data pertaining to the detected cardiac arrhythmia to at least one of: a user interface, a remote server, for further review.

17. The method of claim 1, wherein processing the acquired vibrations-based physiological signals comprises applying a band-pass filter in a 0.5-20 Hz range to attenuate respiratory, motion, and environmental noise components, and applying at least one of adaptive filtering, wavelet transform-based denoising, empirical mode decomposition, and blind source separation using Independent Component Analysis, Principal Component Analysis, or Non-negative Matrix Factorization.

18. The method of claim 1, wherein the heart rate and the heart rhythm is estimated using one or more techniques selected from digital filtering, adaptive thresholding, peak detection, interval analysis, and template matching.
